(19) United States
(12) Patent Application Publication
(10) Pub. No.: US 2001/0005507 A1
(43) Pub. Date: Jun. 28, 2001

(54) Predictive test for hepatitis-B resistance

(75) Inventors: Adrian V.S. Hill, Oxford (GB); Mark R. Thursz, London (GB); Howard C. Thomas, London (GB)

Correspondence Address: Nixon & Vanderhye P.C., 8th Floor, 1100 N. Glebe Rd., Arlington, VA, 22201, US

(73) Assignee: Imperial College of Science, Technology & Medicine

(21) Appl. No.: 09/754,290

(22) Filed: Jan. 5, 2001

Related U.S. Application Data
(63) Continuation of application No. 09/415,232, filed on Oct. 12, 1999, now abandoned.
Application No. 09/415,232 is a continuation of application No. 08/810,380, filed on Mar. 3, 1997, now abandoned.

(30) Foreign Application Priority Data
Sep. 1, 1995 (US) PCT/GB95/02067
Sep. 2, 1994 (GB) 9417850.6

(51) Int. Cl. C07K 1/00; C07K 14/00; C07K 17/00

(52) U.S. Cl. 424/189.1; 530/350; 435/5

(57) ABSTRACT

Methods of identifying resistance to Hepatitis B infection are disclosed, as well as peptides capable of modifying immune response and methods of treating Hepatitis B.

The present invention relates to a novel method of predicting resistance to Hepatitis B, peptides useful in modifying immune response to Hepatitis B, vaccines against Hepatitis B and methods of treating Hepatitis B.

Infection with hepatitis B virus (HVB) may result in a number of disease states ranging from fulminant hepatitis with liver failure to asymptomatic persistent carriage. Most patients will suffer an acute hepatitis during which the virus is eliminated. About 5% of patients in North America and Europe fail to eliminate the virus, whereas in West Africa up to 15% of infected patients fail to clear HVB (Ryder, R. W. et al, Lancet, ii(8400): 449-52 1984). Persistent HBV infection predisposes the host to chronic liver disease and hepatocellular carcinoma (Beasley, R. et al, Lancet, ii, 1159-63, 1981), which is a common cause of death in adults of working age in West Africa (Ryder, R. W. et al., Am. J. Epidemiol., 136(9): 1122-31, 1992).

The outcome of HBV infection does not appear to be determined by variations in virulence of the virus, and the course of disease may be influenced by the host immune response. Although a proportion of patients with persistent infection have specific immunodeficiency states such as HIV (Krosgaard, K. et al, Hepatology, 7: 37-41, 1987) or agamma-globulinaemia (Hermaszewski, R. A. et al, Q. J. Med., 86(1): 31-42, 1993), the majority are not otherwise immunocompromised.

In West Africa, HBV is transmitted via a horizontal route with young children (under 10 years) acquiring the infection from older siblings or playmates (Marinier, E. et al, West Africa J. Pediatr., 106(5): 843-9, 1985; and Botha, J. F. et al, Lancet, i(8388), 1210-2, 1984). Vertical transmission, as seen in the Far East, is rare, so that immaturity of the immune system and the tolerogenic effects of trans-placental HBV ‘e’ antigen (HBeAg) transfer are unlikely to affect the outcome of infection. Epidemiological studies have shown that about 80% of the adult population have been infected with HBV, and that the persistent carriage rate, as determined by HBV surface antigen carriage, is around 12-15% (Ryder et al, supra). Death from HBV related hepatocellular carcinoma is the leading cause of male mortality in adults of working age in The Gambia (Ryder et al, Am. J. Epidemiol., supra; and Kiire, C. F., Vaccine, 8: 5107-112, 1990).

Recognition of foreign antigens by T lymphocytes is achieved through the presentation of antigenic peptides in the groove of MHC encoded HLA molecules. Such immunological responses are MHC restricted, meaning that foreign antigens are only recognised when presented by specific class I or class II molecules. In patients with acute hepatitis B, class I restricted cytotoxic T lymphocytes (CTL) are present in the peripheral blood which recognise the nucleocapsid and envelope antigens of HBV (Bertoletti, A. et al, Proc. Natl. Acad. Sci, USA, 88(23): 10445-9, 1991). In addition there is a strong class II restricted proliferative response to the nucleocapsid antigens (Ferrari, C. et al, J. Clin. Invest, 88: 214-22, 1991). CD4+ T helper cell responses to the nucleocapsid and envelope antigens of the virus are required to support anti-HBe and anti-HBs antibody development (Milich, D. et al, Nature, 319: 547-9, 1987). In patients with chronic HBV infection, CTL are not detectable and the proliferative response is absent or significantly reduced (Ferrari, C. et al, J. Immumol., 145(10): 3442-9, 1990; and Tsai, S. et al, J. Clin. Invest., 89: 87-96 1992).

Several studies have examined the role of MHC phenotype in the outcome of HBV infection without reaching a firm conclusion (Van-Hattum, J. et al, Hepatology, 7 (1): 11-14, 1987; Forzani, B. et al, Hepatology, 4: 1107-10, 1984; Lepage, V. et al, Tissue Antigens, 18: 105-7, 1981; and Kaslow, R. and Shaw, S., Epidemiol. Rev. 3: 90-114, 1981). However, the majority of these studies have been of a size that could only detect a very strong association between MHC phenotype and disease state. Furthermore the sensitivity of these studies has been compromised by using serologically defined MHC class II typing methods which may assign a large number of alleles to the same serological specificity.

DRB1*1302 is associated with a reduced risk of cerebral malaria in Gambian children, which may relate to the high frequency of the DRB1*1302-DRB3*0301-DQA1*0102-DQB1*0501 and DRB1*1302-DRB3*0301-DQA1*0102-DQB1*0604 haplotypes in this population (Lepage, V. et al, supra). The combined haplotype frequency in North Europeans is 4.4% compared with 16.4% in The Gambia (Hill, A. et al, Nature, 352(6336), 595-600, 1991). Malaria and HBV are both important causes of premature mortality in West Africa. However, whereas severe malaria has a high mortality rate in children under 5, HBV related mortalities are common during working life. If recovery from HBV infection were also linked to DRB1*1302, in addition to malaria, HBV would confer a selective advantage on individuals carrying the DRB1*1302 haplotypes in The Gambia.

A recent study has shown a protective effect against human papilloma virus (HPV) related cervical carcinoma associated with DRB1*1302 and DRB1*1301 (Apple, R. J. et al, Nature Genetics, 6(2): 157-162, 1994).

DRw6, which is the serological supertype of DRB1*1302 and DRB1*1301, was identified in two previous studies as potentially protective against persistent HBV infection. Van Hattum et al found DRw6 nearly twice as frequently in North European patients who cleared HBV than in those who failed to eliminate the virus (Van-Hattum, J. et al, supra). In a study of factors influencing the response of chronic HBV infection to interferon therapy, we found that DRw6 is associated with a favourable response (Scully, L. et al, Hepatology, 12: 1111-17, 1990). However, both these studies were too small to reach statistical significance.

It has now been found that the presence of certain HLA molecules confers resistance to infection by Hepatitis B.

Accordingly, the present invention provides a method of identifying resistance to Hepatitis B infection which comprises the step of identifying the presence of HLA-DRB1*1302. Suitably identification is carried out on a sample of blood. Thus, the invention provides a convenient method of predicting Hepatitis B resistance in any given individual and hence also allows for predictions to be made concerning the outcome of Hepatitis B infection in individual patients.

In one embodiment the method of the present invention further comprises the step of identifying the presence of HLA-DRB1*1301. Again, this is suitably carried out by analysing a blood sample.

Furthermore, in view of the recognition that HLA-DRB1*1302 and/or HLA-DRB1*1301 is/are associated with resistance to Hepatitis B, it is possible to modify the immune response of an individual by means of peptides which bind to one or both of those HLA molecules. In another aspect therefore, the present invention also provides one or more peptides capable of binding to HLA-DRB1*1302 and/or HLA-DRB1*1301. Such peptides can be used to modify the ability of HLA-DRB1*1302 and/or HLA-DRB1*1301 to elicit an immune response. Such an approach would be useful in immunisation against HBV infection, and in DRB1*1302 and *1301 individuals, who had become persistently infected, would facilitate recovery (therapeutic immunisation). In a preferred embodiment, binding of the one or more modified peptides will result in a reduced immune response. This is particularly advantageous in treating conditions such as fulminant Hepatitis B.

More generally the present invention provides a peptide capable of modifying the ability of an HLA to elicit an immune response in response to a Hepatitis B antigen. Preferably the peptide is one derived from a Hepatitis B antigen. For example, the peptide can consist only of a particular region known to bind to a particular HLA molecule. Alternatively, a synthetic peptide could be constructed consisting of a binding region and other, non-binding regions.

The peptides can be administered in the form of a pharmaceutical formulation, eg. as an intravenous formulation. Thus, in another aspect the invention provides a pharmaceutical formulation comprising one or more peptides capable of binding to HLA-DRB1*1302 and/or HLA-DRB1*1301, together with one or more pharmaceutically acceptable carriers and/or excipients.

In addition, such peptides can be used in the production of a vaccine against Hepatitis B. Accordingly, the invention also provides a vaccine against Hepatitis B comprising one or more peptides capable of binding to HLA-DRB1*1302 and/or HLA-DRB1*1301.

The peptides of the invention also provide other methods of treating Hepatitis B. For instance, antigen specific lymphocytes can be generated in vitro using a peptide of the invention. These can then be administered to a patient suffering from Hepatitis B. The present invention therefore also provides a composition comprising lymphocytes wherein the lymphocytes have been exposed to one or more peptides of the invention. A method of treating Hepatitis B is also provided which comprises the step of administering such a composition to a subject.

In general the lymphocytes will either be from the subject being treated or from another with a similar HLA type.

The invention will now be described by means of the following example which should not be construed as limiting the scope of the invention in any way.

The example refers to the figures wherein:

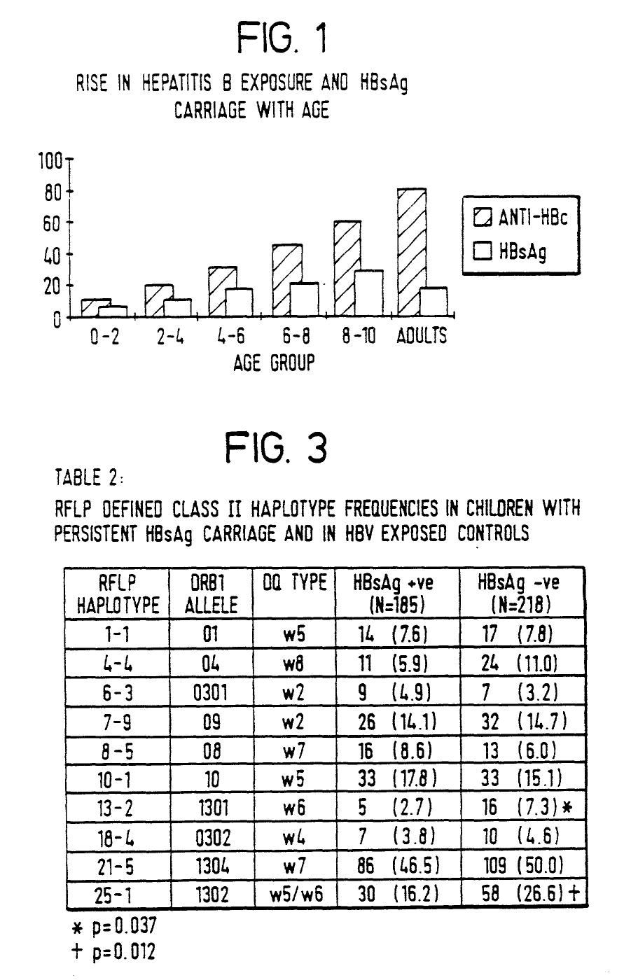

FIG. 1; shows Hepatitis B exposure and HBsAg carriage rise with age during childhood.

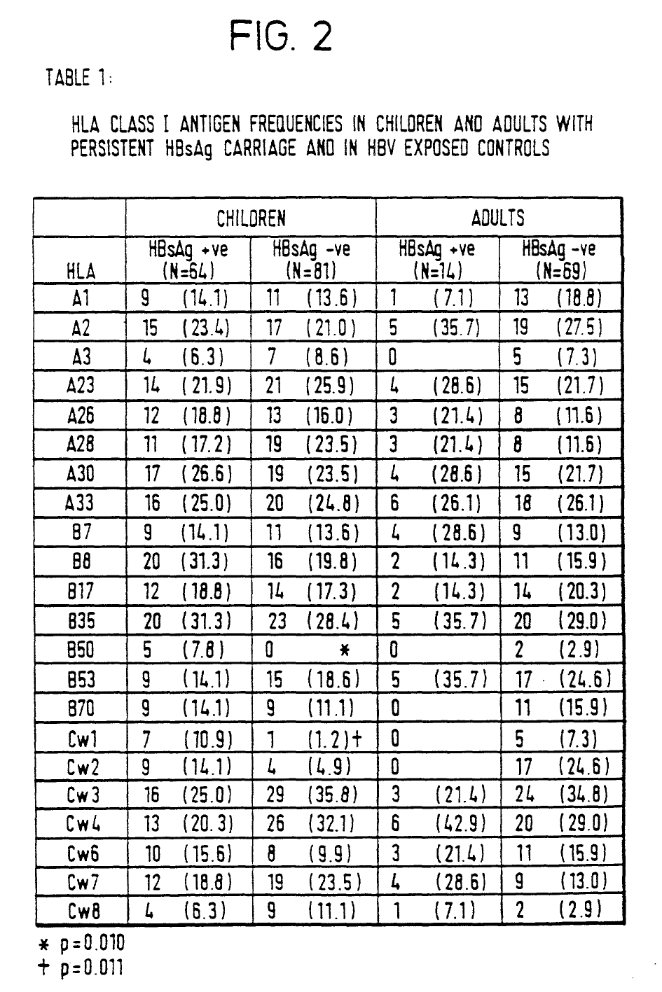

FIG. 2; shows the frequencies of HLA class I serotypes for each group of children and adults.

FIG. 3; shows the class II haplotypes for the children.

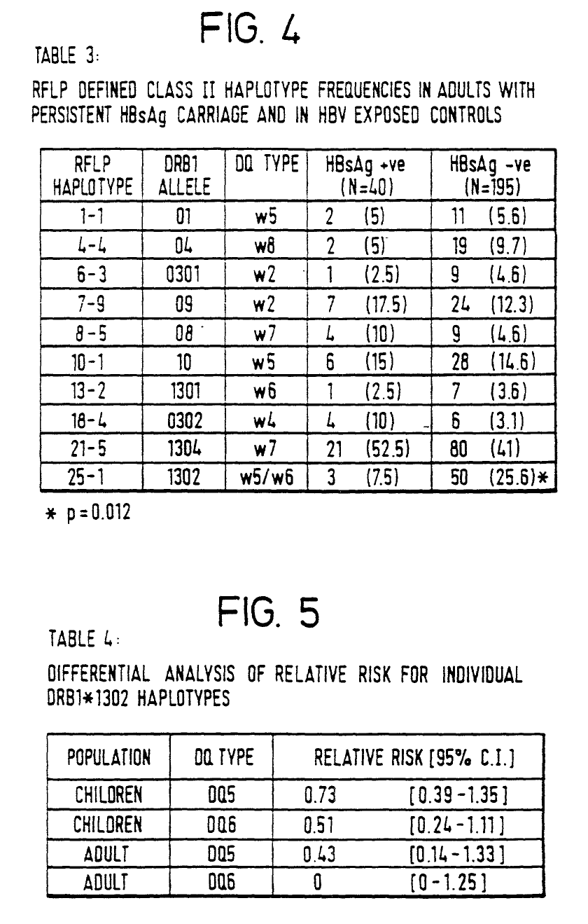

FIG. 4; shows the class II haplotypes for the adults; and

FIG. 5; shows the differential analysis of risk for individual DRB1*1302 Haplotypoes.

### EXAMPLE 1

### Study Populations

The subjects were all Gambians living in the area surrounding the capital, Banjul, which is in the western coastal region. Two different populations were recruited for the two stages of the study between 1988 and 1990. In the first stage, children up to 10 years were recruited at the Royal Victoria Hospital, Banjul, and the Medical Research Council Hospital, Fajara, where they had been seen for a variety of conditions unrelated to HBV. The adult population was recruited from healthy male blood donors. Both populations had previously been studied as part of a case-control study of susceptibility to malaria (Hill , A. et al, supra).

In both stages, subjects were divided into groups according to serological tests for HBV. Group A, who had never been exposed to HBV, were anti-HBV core antibody (anti-HBc) negative. Group B, who had spontaneously recovered from HBV infection, were Anti-HBc positive and HBV surface antigen (HBsAg) negative. Group C, who had persistent HBV infection, were Anti-HBc positive and HBsAg positive. Patients in Group C, who had IgM antibodies to HBV core antigen, were excluded from the analysis. Individuals who had received vaccination against HBV (<10%) fell into group A and were not therefore included in the analysis of HLA frequencies. There were a total of 1344 children; 891 in Group A, 218 in Group B and 185 in Group C. In the Adult population there were a total of 260 subjects: 25 in Group A, 195 in Group B and 41 in Group C. The very small number of individuals with HIV antibodies (<1%) were excluded.

### Serological Testing

Plasma samples were taken from all subjects and stored at −20° C. Anti-HBc, Anti-HBc(IgM) and HBsAg status and anti-HBs antibody concentration were determined by ELISA according to the manufacturers instructions (Boehringer Mannheim, Munich, Germany). Anti-HIV status was determined by Wellcozyme ELISA (Wellcome, Beckenham, UK) and positive results confirmed by Western Blot.

### HLA Typing

Class I serotyping was performed on approximately half the subjects in each population. Serological MHC class I types were determined by standard microlymphocytotoxicity assays using 180 well characterised antisera on fresh or cryopreserved cells (Rood, J., Van, Manual of Tissue Typing Techniques, Bethesda, Md.: National Institutes of Health, 104-105, 1979). MHC Class II typing was performed by restriction fragment length polymorphism analysis, as previously described, using the restriction enzymes Tag1 and BamH1 (Hill et al, supra; and Hill, A. et al, Proc. Natl. Acad. Sci. USA, 89: 227-81, 1992). This was supplemented, where necessary, by polymerase chain reaction amplification and sequence specific oligonucleotide blotting (Hill et al, supra; and Hill, A. et al, Proc. Natl. Acad. Sci. USA, 89, 227-81, 1992). All patients were assigned a class II type.

### Statistical Analysis

Comparisons of phenotype (RFLP-defined haplotype for class II) frequencies between groups B and C were made for 23 HLA class I antigens and 10 class II haplotypes using the χ² test. When multiple comparisons are made concurrently, an apparently significant association may arise by chance. In order to avoid this type of error, we adopted a two stage strategy: multiple comparisons were made in the first population (children), and the second population (adults) was then used to test a single hypothesis (Hill, A., J. RCP., 26(1): 11-16, 1992). Relative risk was calculated by the crude odds ratio and is given with a 95% confidence interval. As there is a mixed ethnic composition in The Gambia (Hill, A. et al, Proc. Natl. Acad. Sci. USA, supra), Mantel-Haenszel tests were performed to assess a possible confounding effect of ethnic origin. Anti-HBs concentration were compared in the adult population between subjects with HLA-DRB1*1302 and those without, using the unpaired t test.

### Results

453 of the 1344 children recruited (33.7%) were anti-HBc positive and, of these, 185 (13.8%) were HBsAg positive and anti-HBc (IgM) negative. 218 (16.2%) children were anti-HBc (total) positive, anti-HBc (IgM) negative and HBsAg negative. The rise with age of anti-HBc and HBsAg carriage are shown in FIG. 1. 260 adults were recruited to the study, of which 235 (90.4%) were anti-HBc positive and 41 (15.8%) HBsAg positive. The frequencies of HLA class I serotypes and class II haplotypes for each group of children are shown in Tables 1, 2 and 3 respectively.

Possible associations between the HLA class I antigens, HLA-B50 and HLA-Cw1 and persistent HBsAg carriage were suggested by the data from the study of children (Table 1). However, these findings were not supported by the data from the adults studied (Table 1). Hence, no class I phenotype was significantly associated with HBV clearance.

HLA class II haplotypes were initially determined using the restriction enzyme Tag1 to define restriction fragment length polymorphism (RFLP) haplotype (Hill, A. et al, Proc. Natl. Acad. Sci. USA, supra). The RFLP pattern 25-1, which corresponds with the class II haplotypes DRB1*1302-DRB3*0301-DQA1*0102-DQB1*0501 (DRw13/DQw5) and DRB1*1302-DRB3*0301-DQA1*0102-DQB1*0604 (DRw13/DQw6), was found in 26.6% of the children who had cleared HBV infection (Group B) and in 16.2% of the children with persistent HBV infection (Group C) (relative risk 0.53 [95% CI 0.32-0.90], p=0.012), and is therefore associated with a protective effect against persistent HBV carriage. The RFLP haplotype 13-2, which corresponds to the class II haplotype DRB1*1301-DRB3*0101-DQA1*0103-DQB1*0603, was also found at a significantly reduced frequency in persistently infected children (p=0.037). No other haplotype showed a significant frequency alteration. We therefore proceeded to analyse these two possible associations in the adult population. The HLA-DRB1*1302 haplotypes were found in 50 of 195 (25.6%) adult subjects from Group B (cleared infection) and 3 of 40 (7.5%) patients with persistent infection (relative risk 0.24, [95% confidence interval 0.04 to 0.80], p=0.012). After stratification by ethnic origin, the Mantel-Haenszel weighted relative risk was 0.23 [95% CI 0.05-0.83], p=0.022; therefore, a significant confounding effect of ethnic origin was excluded. The possible association with HLA-DRB1*1301 was not confirmed by the data on the adult population.

Subjects with the 25-1 haplotypes from both populations were further subdivided into the DQw5 or DQw6 haplotypes. Relative risk and 95% confidence intervals were calculated for both haplotypes and are compared in Table 4. Elimination of HBV is associated with both haplotypes, which suggests that protection from persistent HBV carriage is associated with the HLA-DR molecule bearing DRB1*1302.

In 25 adults with DRB1*1302 (mean age 28.6), the mean anti-HBs concentration was 15.5 iu/L (s.d. 41.5), and in 25 adults without DRB1*1302 (mean age 32.2) the mean anti-HBs antibody concentration was 22.3 (s.d. 48.5); p= 0.598.

### Discussion

This is the first large HLA association study of HBV infection which has incorporated molecular class II analysis. The study clearly shows that the RFLP defined haplotype 25-1 is associated with the ability to clear HBV after infection. The 25-1 haplotype represents two MHC class II haplotypes, DRB1*1302-DRB3*0301-DQA1*0102-DQB1*0501 and DRB1*1302-DRB3*0301-DQA1*0102-DQB1*0604. Further analysis has shown that the two DRB1*1302 haplotypes are both associated with HBV clearance, and it is therefore probable that one or both of the shared HLA-DR alleles are responsible for this association. HLA-DRB1*1302 is only found on the 25-1 RFLP haplotypes, whereas HLA-DRB3*0301 is also found on other RFLP haplotypes in The Gambia. Furthermore, the product of DRB1*1302 is expressed at a higher level than the DRB3 locus product and may thus be of greater functional importance. We conclude that, on the 25-1 haplotype, the DRB1*1302 seems most likely to be of importance in viral clearance. It is possible that the RFLP haplotype 13-2, which corresponds to the class II haplotype DRB1*1301-DRB3*0101-DQA1*0103-DQB1*0603, is also associated with a degree of resistance to HBV persistence in the population, as suggested by the data on persistently infected children. Incidently, the DRB1 allele of this haplotype, DRB1*1301, differs from HLA-DRB1*1302 by just a single amino acid substitution.

HBV-specific CD4+ helper activity, as judged by the proliferative response to HBcAg and HsAg, is markedly reduced in patients with persistent infection in comparison to patients with acute self limiting infection (Ferrari, C. et al, J. Immunol., supra). Through its role in antigen presentation, the class II molecule is considered to be critical in the development of CD4+ helper T cell responses and therefore differential ability of class II molecules to present antigens may manifest as variability in the level of CD4+ help. This is consistent with the detection of a specific class II association with HBV clearance. We infer that, in patients with persistent HBV infection, failure to develop anti-HBe and anti-HBs, and the failure to develop sufficient numbers of active HBV-specific CTL, are at least in part related to the lack of CD4+ help mediated by class II molecules with relatively poor antigen presenting ability.

Failure to develop reasonable levels of anti-HBs following vaccination is associated with the haplotype HLA B8, SC01, DR3 in Caucasians (Egea, E. et al, J. Exp. Med., 173 (3): 531-8, 1991). It seemed possible that the association of HLA-DRB1*1302 with HBV clearance might be reflected in a higher titre of anti-HBs antibody in individuals with this HLA allele. However, levels of anti-HBs antibody in adults who had eliminated HBV were not higher amongst those with the HLA-DRB1*1302 allele, suggesting that the provision of extra “help” for the generation of this antibody response is not the critical mechanism in enhanced HBV clearance in carriers of HLA-DRB1*1302.

HBV infected hepatocytes can be recognised and destroyed by HLA class I restricted CTL (Mondelli, M. et al, J. Immunol., 129(6): 2773-78, 1982; and Pignattelli, M. et al, J. Hepatol., 4: 15-21, 1987). The absence of a strong HLA class I association with HBV clearance may imply that HBV antigens are presented with comparable efficiency by all the class I molecules found in high frequency in The Gambia. In chronic HBV infection, HBV-specific CTL are not readily detectable, which may indicate that the numbers are low or that the CTLs are inactive. One interpretation of our finding of an HLA class II association with HBV clearance is that CTL activity requires strong help from CD4+ T helper cells, the level of which is determined by HLA class II polymorphism (Nonacs, R. et al, J. Exp. Med., 176: 519-29, 1992).

DRB1*1302 appears to be associated with a potent protective effect against three important infectious pathogens, but it is not clear how it might exert this effect. Malaria, HBV and HPV are complex pathogens in which there must be hundreds of potential T cell epitopes with variable MHC restriction elements. The occurrence of an MHC association in any of these diseases suggests that there may only be a small number of epitopes to It is therefore conceivable that polymorphisms of the MHC class I and II loci contribute to the variability in outcome from HBV infection.which a protective immune response is mounted.

1. A method of identifying resistance to Hepatitis B infection which comprises the step of identifying the presence of HLA-DRB1*1302.

2. A method as claimed in claim 1 which further comprises the step of identifying the presence of HLA-DRB1*1301.

3. A method as claimed in claim 1 or claim 2 wherein identification is carried out on a blood sample.

4. A method of predicting the outcome of Hepatitis B infection in a patient which comprises identification of the patient's tissue type.

5. A peptide capable of binding to HLA-DRB1*1302 and/or HLA-DRB1*1301.

6. A peptide as claimed in claim 4 capable of modifying the ability of HLA-DRB1*1302 and/or HLA-DRB1*1301 to elicit an immune response.

7. A peptide capable of modifying the ability of an HLA to elicit an immune response in response to a Hepatitis B antigen.

8. A peptide as claimed in any one of claims 5 to 7 which is derived from a Hepatitis B antigen.

9. A pharmaceutical formulation comprising a peptide as defined in any one of claims 5 to 8, together with one or more pharmaceutically acceptable carriers and/or excipients.

10. A pharmaceutical formulation as claimed in claim 9 which is for intravenous administration.

11. A vaccine against Hepatitis B comprising one or more peptides as defined in any one of claims 5 to 8.

12. A composition comprising lymphocytes which have been treated with one or more peptides as defined in any one of claims 5 to 8.

13. A method of modifying the immune response of a subject to Hepatitis B which comprises the step of administering to the subject one or more peptides as defined in any one of claims 5 to 8.

14. A method as claimed in claim 13 wherein the immune response is reduced.

15. A method as claimed in claim 14 which is used to treat fulminant Hepatitis B.

16. A method for the treatment or prophylaxis of Hepatitis B which comprises the step of administering to a subject one or more peptides any one of claims 5 to 8.

17. A method for the treatment or prophylaxis of Hepatitis B which comprises the step of administering to a subject a pharmaceutical formulation as defined in claim 9 or claim 10.

18. A method for the treatment or prophylaxis of Hepatitis B which comprises the step of administering to a subject a vaccine as defined in claim 11.

19. A method for the treatment or prophlylaxis of Hepatitis B which comprises the step of administering to a subject a composition as defined in claim 12.
